(19) United States
(12) Patent Application Publication
(10) Pub. No.: US 2006/0229358 A1
(43) Pub. Date: Oct. 12, 2006

(54) Pharmaceutical composition containing flavonoids

(75) Inventor: Wun-Chang Ko, Taipei (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: Taipei Medical University, Taipei (TW)

(21) Appl. No.: 11/397,203

(22) Filed: Apr. 4, 2006

(30) Foreign Application Priority Data
Apr. 12, 2005 (TW) 094111580

(51) Int. Cl. A61K 31/353

(52) U.S. Cl. 514/456

(57) ABSTRACT

A pharmaceutical composition is provided, where the pharmaceutical composition contains formula (I), (II), or (III) flavonoids which possess selective phosphodiesterase 4 or 4/3 inhibition, as a main ingredient. Especially, this composition is used in the treatment of asthma, chronic obstructive pulmonary disease (COPD), or chronic inflammation, and has bronchodilatory effects. In addition, whether the above-mentioned flavonoids have side effects, such as nausea, vomiting, gastric hypersecretion, etc., in accordance with their binding to high affinity rolipram binding sites (HARBS) of particulates of brain cells are disclosed.

### FIELD OF THE INVENTION

The present invention relates to a medical composition, and more particularly to a composition including the flavonoid compound, which possess selective inhibition on the phospodiesterase (PDE)4 or 3/4.

### BACKGROUND OF THE INVENTION

PDEs have been classified according to their primary protein and cDNA sequences, co-factor and substrate specificities, and pharmacological roles. Giembycz has disclosed that the PDEs is classified to at least 11 distinct enzyme families that hydrolyze cAMP and/or cGMP [1]. The PDE1-5 isozymes are characterized as being calcium/calmodulin-dependent (PDE1), cGMP-stimulated (PDE2), cGMP inhibited (PDE3), cAMP-specific (PDE4), and cGMP-specific (PDE5) respectively. The PDE1-5 isozymes have been found to be present in canine trachea [2], human bronchi [3], and guinea pig lung [4]. In the guinea pig airway, the PDE3 and PDE4 have been identified, but other isozymes might also be present [5].

It is known that the adenylyl cyclase and the PDEs are mature enzymes responsible for modulating the level of the cytosolic signal transduction material, the cAMP, where the adenylyl cyclase is responsible for the production of the biologically active cAMP from the substrate ATP, and the PDEs are responsible for degrading the biologically active cAMP to the biological inactive molecule 5′-AMP.

The atopic asthma is a chronic inflammatory disorder of the airways. Busse and Lemanske [6], and Maddox and Schwartz [7] have disclosed that atopic asthma is characterized by reversible and recurrent of acute bronchial obstruction and airway hyperreactivity (AHR) with the following mechanism. Once inhaled the antigen in the airway, the antigen would bind to T cells and these cells in turn induce the production of cytokines, such as interleukin (IL)-4, IL-13, and IL-5. IL-4 and IL-13 will subsequently bind with B cells for producing immunoglobulin E (IgE) antibodies. Once synthesized and released by B cells, IgE antibodies briefly circulate in the blood before binding to IgE-bound high-affinity Fc receptors (FcεRI) on the surface of mast cells. And exposing to the allergen once again, the antigen will cross-link with the mast-cell-bound IgE. This cross-linking induces the mast cell to release the chemical cytokines and cysteinyl-leukotrienes that lead to the initiation of the inflammatory reaction of the eosinophils. In addition, Willis-Karp [8] has disclosed that through the release of cytokines IL-5 from T cells, it directly leads to the production of the eosinophils, where the production of the eosinophils in the airway is highly related to the atopic airway inflammatory response, and the production of the eosinophils in the lung erupts with the remodeling of the airway and change the airway tone controlled by the nervous system. Moreover, Kumar [9] has disclosed that the shedding of the epithelium causes the acute bronchial obstruction and airway hyperreactivity (AHR).

Presently, besides the traditional drug for asthma, the aminophylline, the other spray selective agonists, the β-adrenoceptor, for emergency use also releases the bronchial obstruction. However, since frequently using the spray leads to the decrease of the number of the receptors (down-regulation), the curative effect of the β-adrenoceptor will decrease in accordance therewith. The steroids are used for the inflammatory response. However, there are side effects, such as development of moon-face, broader shoulders, adrenal gland atrophy, decrease in immunity, and so on when take steroids for a long time. Moreover, even the spray steroid taken by inhalation also has the problems of Candida albicans infections. Hence, people are looking for new drugs in the treatment of asthma.

Flavonoids are naturally polyphenolic compounds and are widely distributed in the plants and vegetables. It has been reported that the daily diary intake of flavonoids for westerner per day is 1 g. There are approximately 4000 kinds of the naturally polyphenolic compounds in the world, and based on the structures thereof, they are classified into four groups, flavones, flavonols, flavanones and isoflavones. Please refer to Table 1 which shows the structures of the mentioned flavonoids, wherein the respective benzene ring A thereof is condensed with the six-member ring (C), in which the 2-position carries the phenyl benzene ring (B) as a substituent.

TABLE 1
Structures of flavonoids investigated for the
inhibition of the activities of PDE1-5

| 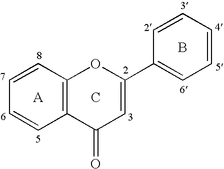 | | | 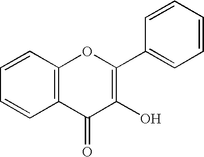 | | | |
|---|---|---|---|---|---|---|
| Flavones | | | Flavonols | | | |
| 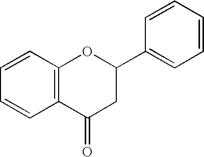 | | | 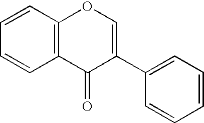 | | | |
| Flavanones | | | Isoflavones | | | |
| | | Substitution | | | | |
| Class | Name | 5 | 7 | 3′ | 4′ | 5′ |
| Flavones | Luteolin | OH | OH | OH | OH | |
| | Luteolin-7- | OH | O-glu | OH | OH | |
| | glucoside | | | | | |
| | Diosmetin | OH | OH | OH | OCH₃ | |
| | Apigenin | OH | OH | | OH | |
| | Chrysin | OH | OH | | | |
| Flavonols | Quercetin | OH | OH | OH | OH | |
| | Myricetin | OH | OH | OH | OH | OH |
| Flavanones | Eriodictyol | OH | OH | OH | OH | |
| | Hesperetin | OH | OH | OH | OCH₃ | |
| Isoflavones | Genistein | OH | OH | | OH | |
| | Daidzein | | OH | | OH | |
| | Biochanin A | OH | OH | | OCH₃ | |
| | Prunetin | OH | OCH₃ | | OH | |

Glu: glucose.

Flavonoids are reported to have anti-inflammatory and immuno-regulatory potentials. Baumann et al have disclosed the inhibition of flavonoids for cyclooxygenase in the cell [10]. Havsteen has disclosed that flavonoids inhibit lipoxygenase and have anti-inflammatory and antioxidant potentials [11]. Also, flavonoids are reported to have anticancer and anti-viral potentials [12], and to have potentials for being an angiogenic inhibitor [13] respectively. In addition, in Raw 264.7 cells, the inhibition of nitric oxide (NO) production and of inducible NO synthase (iNOS) expression by flavonoids has been reported by Kim et al [14]. Moreover, it has been reported that proteoglycan and/or the anti-histamines is administered with or without flavonoids for treating diseases induced by activation of mast cell (such as allergy), though flavonoids are not the principal component [15].

Akiyama et al have disclosed that genistein is a selective inhibitor of tyrosine-specific protein kinase [16]. However, besides being a selective inhibitor of tyrosine-specific protein kinase, genistein with its tyrosine kinase-independent inhibition of cyclic-AMP PDE has been reported by Nichols and Morimoto [17]. Nichols and Morimoto further investigated the inhibition by genistein on the PDE1, 3 and 4, and, reported that genistein is more selective to inhibit PDE4 with the IC₅₀ thereof being 5 μM [18], where no further investigation of the mode of inhibition therefor has been carried out.

Underwood et al have reported that all cyclic AMP-specific PDE4 inhibitors have inhibitory effect on the antigen-induced bronchoconstriction [19]. In addition, Underwood et al also reported that the combination usages of genistein with selective inhibitor of the PDE4 or with the dual PDE3/4 inhibitor effectively inhibit the bronchospasm and the pulmonary eosinophil influx both in vivo and in vitro [20].

Recently, in United States of American and Europe, the selective inhibitors for the PDE4 are considered as the important materials for treating asthma or chronic obstructive pulmonary disease (COPD). In addition, the clinical trials thereof are considered safe and effective, whereas they have side effects, such as vomiting, gastric hypersecretion, etc. [21]. Take the most typical and highly selective PDE4 inhibitor, such as rolipram, for example. In the brain, there are two binding sites for rolipram, which are high (HARBS) and low affinity rolipram binding sites (LARBS), while in the periphery of bronchi and lung there exist only LARBS. Generally, the anti-inflammatory and the bronchodilatory effects of rolipram are considered as its ability to bind with LARBS [21]. The binding ability of rolipram is similar to its ability of inhibition on PDE4 catalytic activity [22], and the side effects are correlated to its binding ability to HARBS [1]. The brain HARBS and peripheral LARBS are called PDE4_{H} and PDE4_{L}, respectively, and the median effective concentration (EC₅₀) of rolipram for binding PDE4_{H} is about 2 nM, and the EC₅₀ of rolipram for binding PDE4_{L} is about 1 μM [23]. Accordingly, the ratio of the EC₅₀ of rolipram for binding PDE4_{H} and PDE4_{L}, which will be called the PDE4_{H}/PDE4_{L} ratio in the following description, is only 0.002, and hence the side effects of rolipram are too big to take it as a therapeutic drug. Therefore, the pharmaceutical factories in the world are trying to develop drugs with high PDE4_{H}/PDE4_{L} ratio for separating the side effects from the main therapeutic effects, while some progresses are obtained. For example, roflumilast has been in clinical trial phase-III for treating both asthma and COPD until 2005, and cilomilast in phase-II for treating asthma until 2003, and in phase-III for COPD, respectively, wherein the PDE4_{H}/PDE4_{L} ratio of roflumilast is 3 [24, 25], and the PDE4_{H}/PDE4_{L} ratio of cilomilast is about 1 [25]. Although the respective PDE4_{H}/PDE4_{L} ratios of roflumilast and cilomilast are much higher than that of rolipram, they are not good enough. AWD-12-281, a newly developed compound with a much higher PDE4_{H}/PDE4_{L} ratio about 11 [26], enters the clinical trial phase-II. It seems having a good perspective.

In order to overcome the foresaid drawbacks, the present invention provides a medical composition including the flavonoid compound, which possesses selective inhibition on the PDE4 or 3/4.

### SUMMARY OF THE INVENTION

In accordance with an aspect of the present invention, a medical composition including a flavonoid compound with the formula (I), (II) or (III), as an active constituent for selectively inhibiting at least one of a PDE4 and a PDE3/4 is provided.

In accordance with another aspect of the present invention, a medical composition for treating the asthma, the chronic obstructive pulmonary disease, or the chronic inflammation is provided, wherein the medical composition includes a flavonoid compound with the formula (I), (II) or (III) as an active constituent, and the flavonoid compound selectively inhibiting at least one of a PDE4 and a PDE3/4. The present invention further provides determining that whether the above-mentioned flavonoid compounds have side effects, such as vomiting, gastric hypersecretion, etc., is in accordance with whether the above-mentioned flavonoid compounds bind to the particulate HARBS of the brain cells.

The above objects and advantages of the present invention will become more readily apparent to those ordinarily skilled in the art after reviewing the detailed description of the preferred embodiments of the present invention and accompanying drawings.

The mentioned flavonoid with the formula (I), (II) or (III) of the present invention, preferably, hesperetin, prunetin and the derivatives thereof have the high PDE4_{H}/PDE4_{L} ratio. According to the preliminary experimental data of the present inventor, hesperetin or prunetin even at a higher concentration (300 μM) bound to HARBS of the whole brain cell particulates in guinea pig only 17.5% and 24.2%, respectively. That is to say, for having 50% of HARBS been bound by hesperetin or prunetin, the concentration thereof shall be more than 300 μM (the concentration-over 300 μM is not available due to the solubility thereof). Since hesperetin is a kind of flavanones and prunetin is one of isoflavones, therewith little side effects are expectable. The respective IC₅₀ values of hesperetin and prunetin for PDE4 are 28.2 μM and 61.9 μM, respectively [27]. Therefore, their concentrations for binding to an half of the LARBS could be considered as 28.2 μM and 61.9 μM [22]. Accordingly, the PDE4_{H}/PDE4_{L} ratio for hesperetin is certainly greater than 11, and the ratio for prunetin is greater than 5 at least. Hence, hesperetin and prunetin with little side effects, are hopeful to be the effective drugs against asthma, COPD and inflammation (including airway inflammation, arthritis and rheumatoid arthritis).

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1A is a drawing showing the cAMP hydrolysis enzyme activity for PDE 4 in relation to the treatment of hesperetin at various concentrations;

FIG. 1B is a drawing showing the cAMP hydrolysis enzyme activity for PDE 4 in relation to the treatment of rolipram at various concentrations;

FIG. 2 is a drawing showing the percentage of displacement of [³H]-rolipram on HARBS in relation to the treatment of different drugs at various concentrations;

FIG. 3A is a drawing showing the enhanced pause in relation to hesperetin at various doses administered in animal model;

FIG. 3B is a drawing showing the inflammatory cells from BALF in relation to hesperetin at various doses administered in animal model;

FIG. 3C is a drawing showing the cytokines from BALF in relation to hesperetin at various doses administered in animal model; and

FIG. 4 is a drawing showing percentage of the cumulative OVA-induced contractions, standardized by 60 mM KCl, in relation to the inhibitory effects of hesperetin preincubated at various concentrations in vitro.

### DETAILED DESCRIPTION OF THE PREFERRED EMBODIMENT

The invention is described more specifically with reference to the following embodiments. It is to be noted that the following descriptions of preferred embodiments of this invention are presented herein for the purposes of illustration and description only; it is not intended to be exhaustive or to be limited to the precise form disclosed.

Reagents and Drugs

Hesperetin, other flavonoides listed in the following Table 1, Bis-tris base, Trizma base, D,L-dithiothreitol, benzamidine, EDTA, EGTA, PMSF, BSA, cyclic AMP, cyclic GMP, calmodulin, Dowex resin, DMSO, and Crotalus atrox snake venom, etc. were purchased from Sigma Chemical (St. Louis, Mo., USA). [³H]cAMP, [³H]cGMP, Q-sepharose, and calmodulin-agarose were purchased from Amersham Pharmacia Biotech (Buchinghamshire, UK). Vinpocetin, EHNA, Ro 20-1724, milrinone and zaprinast were purchased from Biomol (Plymouth Meeting, Pa., USA). Ethyleneglycol was purchased from Merck (KgaA, Darmstadt, Germany). Prunetin were purchased from Fluka Chemie (Gmbh CH-9471 Buchs, Switzerland). Other reagents, such as CaCl₂, MgCl₂, and NaCl, were of analytical grade.

The above-mentioned genistein, daidzein, biochanin A, prunetin and vinpocetin were dissolved in a mixture of DMSO and ethyl alcohol (1:1). EHNA, Ro 20-1724, and PMSF were dissolved in ethyl alcohol. Milrinone and zaprinast were dissolved in DMSO. EGTA was dissolved in 3N NaOH. Other drugs were dissolved in distilled water. The respective final concentrations of solvents were less 0.1% and did not significantly affect the activities of the PDE isozymes. All drug concentrations were presented in molarity.

Separation of Cyclic Nucleotide PDE Isozymes

Under a protocol approved by the Animal Care and Use Committee of Taipei Medical University, five male guinea pigs (Hartley), weighing 500-600 g, were sacrificed. The lungs (15 g) or hearts (4 g) taken therefrom were chopped into small pieces and homogenized with a glass/teflon homogenizer (Glas-Col, Terre Haute, Ind., USA) in 10 volumes of cold medium (pH 6.5) containing 20 mM Bis-Tris, 2 mM benzamidine, 2 mM EDTA, 50 mM sodium chloride, 0.1 mM phenylmethanesulfonyl fluoride (PMSF), and 1 mM dithiothreitol. At 4° C., the homogenate was centrifuged at 170 g for 15 min, and the supernatant thereof was then re-centrifuged at 40,000 g for 30 min. The final supernatant fraction was filtered through the 0.22 μm filter and applied to a Q-sepharose fast flow column (2.2×28 cm), which is pre-washed and pre-equilibrated in homogenization buffer. The column was washed with two bed volumes of homogenate buffer to remove unbound material, where the resin beads therein will bind with proteins, such as PDE proteins. Proteins bound to the Q-sepharose beads were eluted with various concentrations (0.23, 0.34, 0.44, 0.69, and 1.00 M) of NaCl dissolved in homogenate buffer (for each concentration, 40-50 mL elution buffer were applied) at a flow rate of 30 ml/h. Fractions (3 ml each) were collected, and ethylene glycol (EG) was added thereto until a final concentration of 30% (v/v). And then the samples were frozen at −70° C. Under these conditions, the enzyme activity was stable for at least 3 months [28].

In order to eliminate possible contamination of PDE5 by PDE1, the above-collected fractions with enzyme activity was further purified on a calmodulin-agarose column. The column (1.6 cm×4 cm) was pre-equilibrated by a buffer A, which contains 20 mM Bis-tris, 1 mM dithiothreitol, 2 mM benzamidine, 50 mM NaCl, 3 mMMgCl₂, 0.1 mM CaCl₂, and 0.1 mM PMSF, pH 6.5. The sample (the mixture of PDE5 and PDE1), being purified by the above-mentioned column, with the CaCl₂ concentration thereof adjusted to at least 2 mM previously, was loaded and allowed to be absorbed on the gel for 30 min. Then, the PDE isozymes were eluted by stepwise elution using 20 ml of buffer A with 1 M NaCl followed by 20 ml of buffer A with 1 M NaCl and with 1 mM EGTA. PDE5 was collected first followed by PDE1.

Assay I: Competitive Inhibition of Flavonoids on Cyclic Nucleotide PDE Activity

The activities of PDE1-5 in the homogenate were measured with a two-step procedure according to the method of Thompson and Appleman [29], using cAMP with [³H]-cAMP or cGMP with [³ H]-cGMP as substrates. The PDE enzyme prepared (25 μl) with 10 μl inhibitor or the solvents therefor was incubated for 30 min at 37° C. in a total assay buffer, where the final volume of the assay is amounted to 100 μl. In accordance with the features of the PDE isozymes, the assay buffer contains 50 mM Tris/HCl (pH 7.4), 3 mM MgCl₂, 1 mM dithiothreitol, and 0.05% BSA, and optionally contains 1 uM cAMP with 0.2 μCi [³H]-cAMP as a substrate alone or in the presence of 0.1 unit calmodulin with 10 μM CaCl₂, or 5 μM cGMP, and 1 μM cGMP with 0.2 μCi [³H]-cGMP as another substrate alone or in the presence of 0.1 unit calmodulin with 10 μM CaCl₂. In assay of enzyme inhibition, the assay mixture contained with the inhibitors at various concentrations of flavonoids in the Table 1 or the selective PDE1-5 inhibitors as reference drugs. For example, the selective PDE1-5 inhibitors are vinpocetin [30], EHNA [31], milrinone [32], Ro 20-1724 [33], and zaprinast [34], respectively.

The PDE enzyme and the inhibitors (or their solvent) therefor were mixed and incubated on ice for 30 min previously, and then mixed with the assay buffer. The assay was initiated by transferring the mixture to a water bath at 37° C. Following the other 30 min incubation, the reaction was stopped by transferring the reaction vessel to a bath of boiling water for 3 min. After cooling on ice, 20 μl of the 1 mg/ml of Crotalus atrox venom was added to the reaction mixture, and the mixture was incubated at 37° C. for 10 min. The uncatalyzed substrates, such as cAMP, [³H]-cAMP, cGMP, or [³H]-cGMP were removed by the addition of 500 μl of a 1-in-1 Tris-HCl (40 mM) buffer suspension of Dowex resin (1×8-200) with incubation on ice for 30 min, since the bindings of the cyclic nucleotides and the resin. Each tube was then centrifuged for 2 min at 6000 rpm, and 100 μl of the supernatant was removed and counted by a β-counter for calculating the enzyme (PDEs) activity. Less than 10% of the tritiated cyclic nucleotide ([³H]-cAMP or [³H]-cGMP) was hydrolyzed in this assay.

The median inhibition concentrations (IC₅₀s) of flavonoides for PDE1-5 activities are listed in the Table 2, wherein hesperetin and prunetin exhibited more-selective inhibition on PDE4.

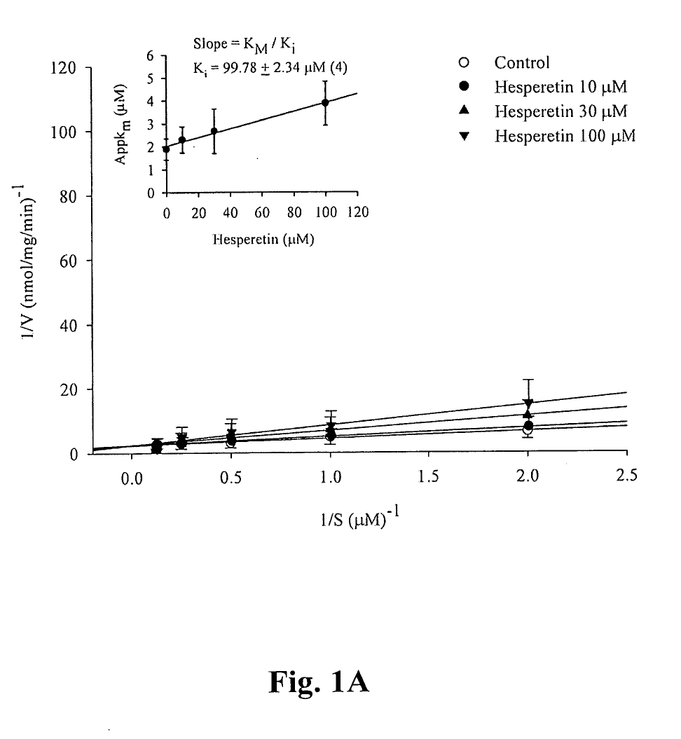

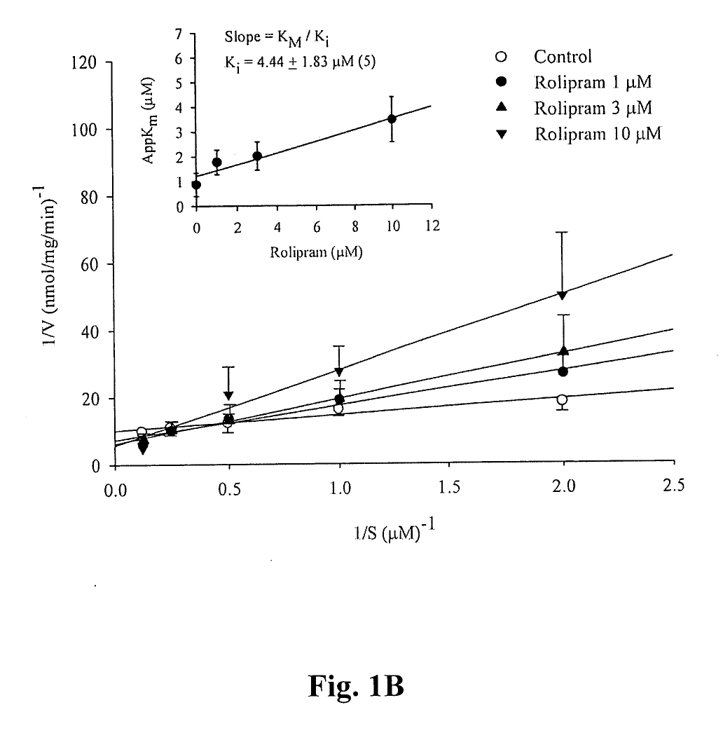

Please refer to FIGS. 1A and 1B, which show the enzyme activity of PDE 4 for cAMP hydrolysis in the incubation of hesperetin and rolipram at various concentrations, respectively. While the activities of PDE4 (reaction velocity, shown as V) in the presence of various concentrations of hesperetin or rolipram and cAMP (substrate, shown as S) were analyzed in accordance to a bouble reciprocal plot, also called a Lineweaver-Burk plot [28]. The mode of action (such as competitive or non-competitive to PDE4) of hesperetin or rolipram was analyzed according to the plot. The dissociation constant of inhibitor binding (Kᵢ) value was determined from the equation of apparent Kₘ as a function of the inhibitor concentration (insert in A and B, respectively). The slope of the equation is equal to the value of K_{M}/Slope (where K_{M} is Michaelis constant). The amounts of the total proteins are calculated based on the analytical method reported by Bradford [35]. In FIGS. 1A and 1B, all enzyme activities are shown as nmole of substrate per mg of proteins per minute (nmole/mg/min) hydrolyzed. As shown in FIGS. 1A, hesperetin competitively inhibits the enzyme activity of PDE4.

TABLE 2
IC₅₀ (μM) values of flavonoids on phosphodiesterase isozymes

| | | PDE isozymes | | | | |
|---|---|---|---|---|---|---|
| Class | Name | 1 | 2 | 3 | 4 | 5 |
| Flavones | Luteolin | 21.5 ± 2.9 (3) | 13.3 ± 0.8 (3) | 10.1 ± 1.8 (5) | 19.1 ± 2.4 (6) | 19.3 ± 3.2 (4) |
| | Luteolin-7-glucoside | >100 (3) | 35.1 ± 0.2 (3)* | >100 (3) | 43.0 ± 5.3 (4)* | >100 (3) |
| | Diosmetin | 14.4 ± 6.2 (3) | 4.8 ± 0.8 (4)*^{,#} | >100 (3) | 20.2 ± 2.4 (3) | 15.3 ± 3.6 (3) |
| | Apigenin | 25.4 ± 3.7 (3)^{#} | 16.7 ± 6.3 (5)^{#} | 10.5 ± 3.5 (4)^{#} | >100 (3) | >100 (3) |
| | Chrysin | >100 (3) | >100 (4) | >100 (3) | >100 (4) | >100 (4) |
| Flavonols | Quercetin | 27.8 ± 5.7(3)^{#} | 17.9 ± 3.4 (4) | 5.6 ± 1.0 (4) | 9.9 ± 2.5 (3)* | >100 (3) |
| | Myricetin | 24.9 ± 3.6 (3)^{#} | 12.8 ± 0.6 (4)^{#} | 12.4 ± 3.3 (4)^{#} | 39.8 ± 2.1(6)*^{,&} | >100 (3)^{#} |
| Flavanones | Eriodictyol | >100 (3) | >100 (3) | 52.5 ± 17.7 (4)*^{,#} | >100 (3) | >100 (3) |
| | Hesperetin | >100 (3)^{#} | >100 (3)^{#} | >100 (3)^{#} | 28.2 ± 1.1(3)^{$} | >100 (3)^{#} |
| Isoflavones | Genistein | 16.8 ± 2.3 (3)^{#} | 1.7 ± 0.2 (4)*^{,#} | 12.9 ± 5.2 (3) | 9.5 ± 1.9 (4)*^{,+} | 73.9 ± 7.1 (3)*^{,#,+} |
| | Daidzein | >100 (3)*^{,!} | >100 (4)*^{,!} | 28.6 ± 8.5 (3)*,# | >100 (4)*^{,!} | >100 (3)*^{,!} |
| | Biochanin A | 29.1 ± 0.3 (3)^{#,}*^{,!} | 27.9 ± 4.1(4)^{#,}*^{,!} | >100 (3)^{#,}*^{,!} | 8.5 ± 0.1 (4)* | >100 (3)^{#,}*^{,!} |
| | Prunetin | >100 (3)*^{,!} | >100 (4)*^{,!} | >100 (3)*^{,!} | 61.9 ± 17.3 (4)*^{,!} | >100 (3)*^{,!} |
| | Reference drugs^{a} | 122.8 ± 44.9 (3) | 4.4 ± 1.0 (5) | 2.4 ± 0.5 (3) | 11.4 ± 1.6 (8) | 3.3 ± 0.9 (7) |

All values are expressed as the mean ± S.E.M. (n), where n is the number of experiments.
^{a}Reference drugs for PDE isozymes 1, 2, 3, 4, and 5 were vinpocetine, EHNA, milrinone, Ro 20-1724, and zaprinast, respectively.
*p < 0.05 when compared with the corresponding value of luteolin.
^{#}p < 0.05 when compared with the corresponding value of PDE4.
^{$}p < 0.05 when compared with the corresponding value of diosmetin.
^{&}p < 0.05 when compared with the corresponding value of quercetin.
⁺p < 0.05 when compared with the corresponding value of apigenin.
^{!}p < 0.05 when compared with the corresponding value of genistein.

Assay II: The Binding of Flavonoids to Particulate HARBS of Guinea Pig's Whole Brain

The binding experiments are carried on basis of the methods of Schneider et al. [23] and Zhao et al. [25] with a small modification. Under a protocol approved by the Animal Care and Use Committee of Taipei Medical University, five male guinea pigs (Hartley), weighing 500-600 g, were anesthetized. The whole brains taken therefrom were homogenized with in 10 volumes of cold medium (pH 6.5) containing 20 mM Bis-Tris, 2 mM benzamidine, 2 mM EDTA, 50 mM sodium chloride, 0.1 mM phenylmethanesulfonyl fluoride (PMSF), and 1 mM dithiothreitol. At 4° C., the homogenate was centrifuged at 170 g for 15 min for removing blood vessels and connective tissues, and the supernatant thereof was then re-centrifuged at 40,000 g for 30 min for separating the particulates from the cytosol of cells. The precipitated particulates were washed with fresh homogenate (4° C.) for several times, and the particulates were re-suspended to a 366 mg/ml of suspension (wet weight of brain per ml), where most of the particulates contained therein are cell membrane. It was found that there are 1.33 fmole [³H]-rolipram binding HARBS per mg of cell membrane after analysis by using Scatchard plots.

The respective binding abilities of the test drugs (flavonoids) to membrane HARBS are performed in a 25 μl of reaction solution, containing 10 μl of [³H]-rolipram, 10 μl of particulate suspension, and 5 μl of test drugs or selective PDE4 inhibitors for 60 min at 30° C., wherein the reaction buffer contained 50 mM Tris-HCl, 5 mM MgCl₂ (pH 7.5). The final concentration of [³H]-rolipram was 2 nM. Whereas, those of test drugs were ranged from 3-300 μM, and those of reference drugs (positive control), non-radioactive rolipram and Ro 20-1724, were ranged from 0.3-1000 nM and 1-10000 nM, respectively. After incubation, the reaction was stopped by transferring the reaction vessel to a bath of crashed ice. Then the reaction mixture was filtered through a punched glass fiber filter (Whatman GF/B) placed in a mini funnel, which was adopted to a 1.5 ml of Eppendorff's tube for collecting the filtrate in the mini centrifuge at 1000 rpm for 10 seconds. By the same way (centrifugation), it is further washed with 0.3 ml reaction buffer each for three times. The Whatman GF/B filter was mixed with 2 ml of cocktail and counted by the β-scintillation counter (Backman, Fullerton, Calif., USA) for counting the radioactivity thereof.

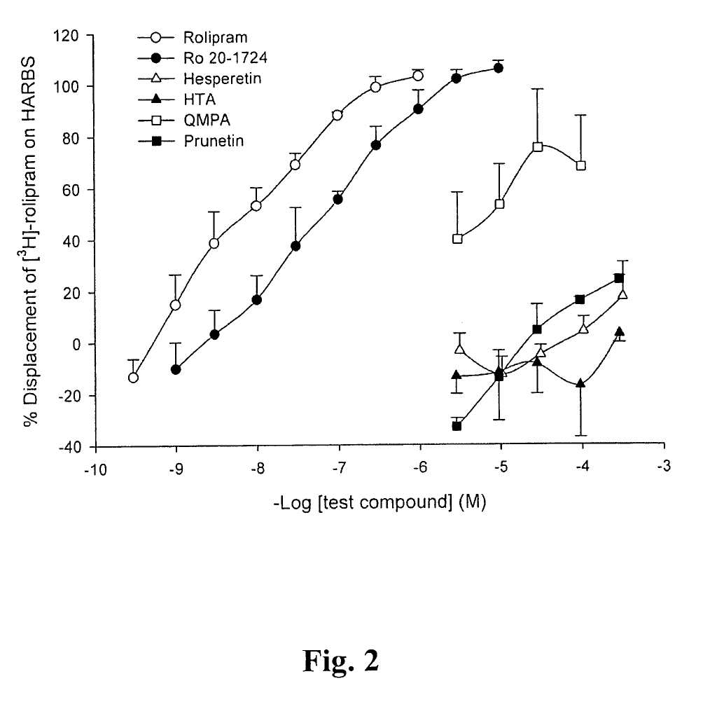

Please refer to FIG. 2, which shows the percentage of displacement of [³H]-rolipram from HARBS by different test drugs at various concentrations. As shown in FIG. 2, the replacement of [³H]-rolipram from HARBS by rolipram or Ro 20-1724 was in a concentration-dependent manner, with the IC₅₀ values thereof being 7.4 and 67.3 nM, respectively. Quercetin 3-O-methyl peracetate (QMPA) also significantly displaced the [³H]-rolipram from HARBS with an IC₅₀ value of 8.7 μM. However, the IC₅₀ values of hesperetin, hesperetin triacetate (HTA), and prunetin are greater than 300 μM.

Assay III: Determination of Airway Hyperresponsiveness (AHR), Inflammation and Cytokines in Vivo

Sensitization, airway challenge, and allergen provocation was carried out as described by Kanehiro et al [36] with some modifications. The female BALB/c mice from 8 to 12 week of age were obtained from National Laboratory Animal Center (Taiwan), and maintained on diets free of ovalbumin (OVA). Mice were sensitized by an intraperitoneal (i.p.) injection of 20 μg of OVA emulsified in 2.25 mg aluminum hydroxide gel in a total volume of 100 μl on days 0 and 14. For comparison, some mice were not sensitized, but received sham injection. All mice were challenged via the airway by OVA (1% in saline) for 30 min on days 28, 29, and 30 by ultrasonic nebulization. Six weeks after the last of the three primary OVA challenges, mice were exposed to 1% OVA for 30 min by nebulization as a secondary challenge [36]. AHR was assessed on day 73 (48 h after 1% OVA provocation) for each group. Each group of mice was intraperitoneally injected with vehicle or hesperetin at 3-30 μmol/kg 2 h before and 6 and 24 h after OVA provocation. The vehicle, a mixture of alcohol: polyethyleneglycol 400: saline (1:14.5:14.5), or hesperetin was injected at a volume of 0.01 ml/g of body weight. The AHR was measured in unrestrained animals by barometric plethysmography [37] using a whole-body plethysmograph (WBP) (Buxco, Troy, N.Y., USA). Mice were placed into the main chamber of the WBP and nebulized first with phosphate buffer solution (PBS), then with increasing doses (6.25-50 mg/ml) of methacholine (MCh) for 3 min for each nebulization, followed by readings of breathing parameters for 3 min after each nebulization with determination of enhanced pause (Penh) values. Twenty-four hours after the Penh determination, these mice were anesthetized with pentobarbital (50 mg/kg, i.p.), and lavaged via a tracheal tube with PBS (1×1.0 ml, 37° C.), under a protocol approved by the Animal Care and Use Committee of Taipei Medical University. The collected bronchoalveolar lavage fluid (BALF) was centrifuged at 500 rpm for 5 min, and the pellet was resuspended in ACK lysing buffer to lyse residual erythrocytes in each sample. The number of inflammatory cells was counted using a hemacytometer (Hausser Sci., Horsham, Pa., USA). Cytospin slides were stained and differentiated in a blinded fashion by counting at least 100 cells under light microscopy. However after centrifugation, the supernatant was stored at −20° C. until the determination of cytokines, including interleukine (IL)-2, IL-4, IL-5, tumor necrosis factor (TNF)-α, and interferon (IFN)-γ by flow cytometric methods using mouse Th1/Th2 cytokine CBA kits (Pharmingen, San Diego, Calif., USA) according to the recommendations of the manufacturer.

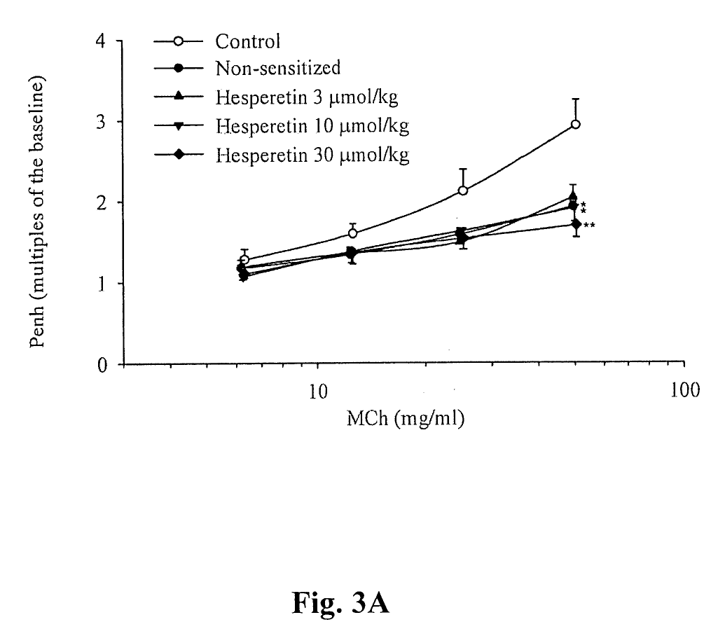

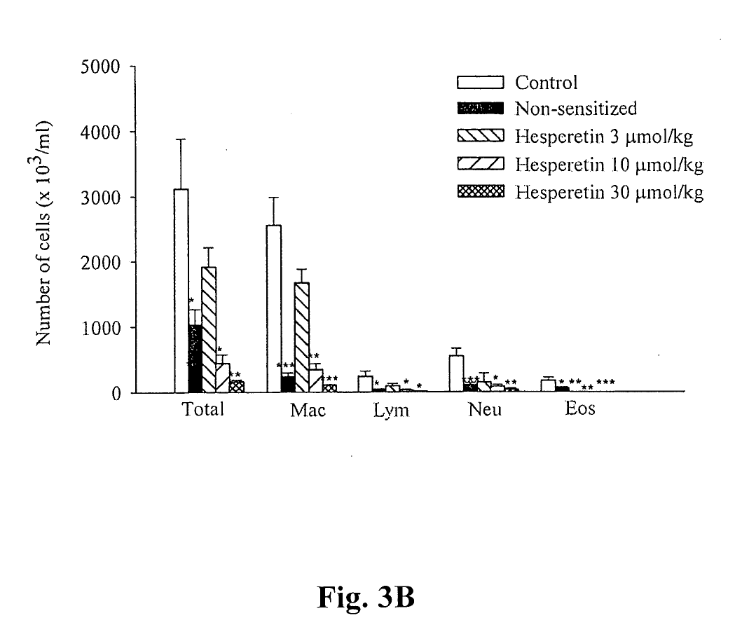

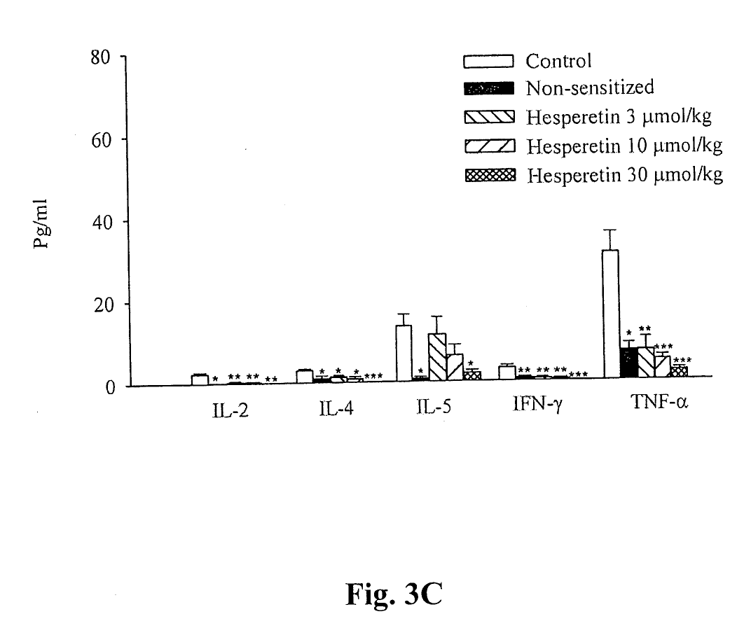

Please refer to FIG. 3A, which shows the airway hyperresponsiveness (AHR) reaction in animal model. It was found that the Penh values largely increased in a concentration dependent manner after exposure in MCh (6.25-50 mg/ml) in the sensitized mice, but only slightly did in non-sensitized ones, and significantly differed from each other. Hesperetin (10 and 30 μmol/kg, i.p.) significantly suppressed Penh values induced by 50 mg/ml MCh. However, all Penh values of hesperetin (3˜30 μmol/kg) were not significantly different from that of non-sensitized mice, suggesting that hesperetin may have potential for treatment of allergic asthma.

FIG. 3B shows the inflammatory cells in BALF of the animal model. The total inflammatory cells, macrophages, lymphocytes, neutophils, and eosinophils in BALF of sensitized mice significantly enhanced from those of non-sensitized mice. Hesperetin at 10 and 30 μmol/kg (i.p.) significantly suppressed these enhancements. Hesperetin even at 3 μmol/kg significantly suppressed the enhancement of eosinophils when compared with control group, suggesting that hesperetin may have anti-inflammatory effects.

FIG. 3C shows the cytokines in BALF of the animal model. The levels of interleukin (IL)-2, IL-4, IL-5, tumor necrosis factor (TNF)-α, and interferon (IFN)-γ in BALF of sensitized mice significantly enhanced from those of non-sensitized mice. Hesperetin (3˜30 μmol/kg, i.p.) significantly suppressed these enhancements, suggesting that it may suppress the productions of cytokines from T helper cells type 1 (Th1) and type 2 (Th2), with an exception that the level of IL-5, which mainly releases from Th2, was not suppressed by hesperetin at 3 and 10 μmol/kg.

Assay IV: Determination of Airway Hyperresponsiveness (AHR) in Vitro

Male Hartley guinea pigs (500-600 g), obtained from National Laboratory Animal Center (Taiwan), were sensitized by intramuscularly injections of 0.35 ml of a 10% (w/v) OVA with Freund's adjuvant into each thigh (total, 0.7 ml) on days 1 and 4. The guinea pigs were ready for use after day 25 [20]. Under a protocol approved by the Animal Care and Use Committee of Taipei Medical University, the tracheae of guinea pigs (Hartley) were sacrificed and removed therefrom. The tracheae were carefully trimmed off the excessive surrounding tissues and cut into six segments. Each segment consisted of three cartilage rings. All segments were cut open opposite the trachealis. After the segments were randomized to minimize regional variability, they were tied at one end to holders via silk sutures, placed in 5 ml of normal Krebs solution containing indomethacin (3 μM), gassed with a 95% O₂ plus 5% CO₂ mixture at 37° C., and attached by the other end of each segment to force displacement transducers (Grass FT03) for the isometric recording of tension changes on a polygraph (Gould RS3200). The composition of the normal Krebs solution was (mM): NaCl 118, KCl 4.7, MgSO₄ 1.2, KH₂PO₄ 1.2, CaCl₂ 2.5, NAHCO₃ 25, and dextrose 10.1. The tissues were suspended in normal Krebs solution under an initial tension of 1.5 g and allowed to equilibrate for at least 1 h with washing at 15-min intervals. After the tissues were precontracted with KCl (60 mM) and washed with normal Krebs solution, OVA (0.1-100 μg/ml) was cumulatively added, and contractions were allowed to reach a steady state at each concentration. To evaluate the suppressive effect of hesperetin on OVA-induced contractions, each tissue was preincubated with each concentration (3, 10, or 30 μM) of hesperetin or its vehicle (DMSO: alcohol, 1:1) for 15 min and then challenged with cumulative OVA again. Therefore, the log concentration-response curves of OVA were constructed in the absence and presence of hesperetin. The tension of precontraction induced by KCl before experiment was set as 100%.

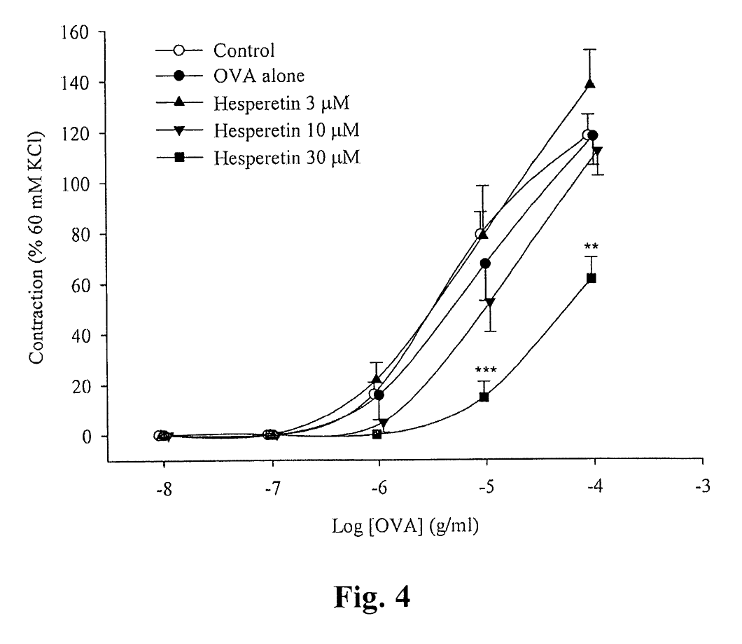

Please refer to FIG. 4, which shows the inhibition by the drug on the cumulative OVA-induced contractions. The experiment was carried out in the presence of hesperetin (3˜30 μM) or the solvent thereof (DMSO: alcohol=1:1) for 15 min, followed by an addition of cumulative OVA (0.0˜1100 μg/ml), and isometrically recorded the contractile responses. The inhibition by hesperetin on the OVA-induced contractions was expressed as a percentage, standardized by 60 mM KCl as 100% before experiment.

As shown in FIG. 4, hesperetin antagonized the OVA (100 μg/ml)-induced contractions in a concentration-dependent manner. Hesperetin at 30 μM significantly antagonized the OVA (both 10 and 100 μg/ml)-induced contractions. Hesperetin, therefore, could inhibit the degranulation of mast cell for releasing the endogenous chemical mediators, such as histamine, prostaglandins, leukotriens, and cytokines.

Statistical Analysis

Concentrations of flavonoids at which 50% of maximum activity (IC₅₀ value) was produced were compared to each other. The IC₅₀ values of flavonoids and various reference drugs were calculated using non-linear regression analysis by the software SigmaPlot 7.0 (Sigma Chemical, St. Louis Mo., USA). All values are shown as the mean±S.E.M. Differences among values, which are equal or greater to three groups, were statistically calculated by one-way analysis of variance (ANOVA), and then determined by the least significant difference (LSD). The difference between two values, however, was determined by use of Student's unpaired t-test. Differences were considered statistically significant if the P-value was less than 0.05.

While the invention has been described in terms of what are presently considered to be the most practical and preferred embodiments, it is to be understood that the invention need not be limited to the disclosed embodiment. On the contrary, it is intended to cover various modifications and similar arrangements included within the spirit and scope of the appended claims which are to be accorded with the broadest interpretation so as to encompass all such modifications and similar structures. Therefore, the above description and illustration should not be taken as limiting the scope of the present invention which is defined by the appended claims.

### REFERENCES

[1] Giembycz M A. Phosphodiesterase 4 inhibitors and the treatment of asthma. Drugs 2000; 59: 193-212

[2] Torphy T J, Cieslinski L B. Characterization and selective inhibition of cyclic nucleotide phosphodiesterase isozymes in canine tracheal smooth muscle. Molecular Pharmacology 1990; 37: 206-214

[3] De Boer J, Philpott A J, van Amsterdam R G, Shahid M, Zaagsma J, Nicholson C D. Human bronchial cyclic nucleotide phosphodiesterase isoenzymes: biochemical and pharmacological analysis using selective inhibitors. British Journal of Pharmacology 1992; 106: 1028-1034

[4] Kapui Z, Schaeffer P, Mikus E G, Boronkay E, Gyürky J, Herbert J M, Pascal M. Experimental studies on guanosine 3′,5′-cyclic monophosphate levels and airway responsiveness of the novel phosphodiesterase type 5 inhibitor SR 265579 in guinea-pigs. Arzneimittel-Forschung 1999; 49: 685-693

[5] Silver P J, Hamel L T, Perrone M H, Bentley R G, Bushover C R, Evans D B. Differential pharmacologic sensitivity of cyclic nucleotide phosphodiesterase isozymes isolated from cardiac muscle, arterial and airway smooth muscle. European Journal of Pharmacology 1988; 150: 85-94

[6] Busse W W, Lemanske R F Jr. Asthma. New England Journal of Medicine 2001; 344: 350-362

[7] Maddox L, Schwartz D A. The pathophysiology of asthma. Annual Review of Medicine 2002; 534: 477-498

[8] Wills-Karp M. Immunologic basis of antigen-induced airway hyperresponsiveness. Annual Review of Immunology 1999; 17: 255-281

[9] Kumar R K. Unerstanding airway wall remodeling in asthma: a basis for improvements in therapy? Pharmacology and Therapeutics 2001; 91.: 93-104

[10] Baumann J, von Bruchhausen F, Wurm G. Flavonoids and related compounds as inhibitors of arachidonic acid peroxidation. Prostaglandins 1980; 20: 627-639.

[11] Havsteen B. Flavonoids, a class of natural products of high pharmacological potency. Biochemical Pharmacology 1983; 32: 1141-1148.

[12] Wang H K, Xia Y, Yang Z Y, Natschke S L and Lee K H. Recent advances in the discovery and development of flavonoids and their analogues as antitumor and anti-HIV agents. Advances in Experimental Medicine & Biology 1998; 439: 191-225.

[13] Fotsis T, Pepper M S, Aktas E, Breit S, Rasku S, Adlercreutz H, Waehaelae K, Montesano R and Schweigerer L. Flavonoids, dietary-derived inhibitors of cell proliferation and in vitro angiogenesis. Cancer Research 1997; 57: 2916-2921.

[14] Kim H K, Cheon B S, Kim Y H, Kim S Y and Kim H P. Effects of Naturally Occurring Flavonoids on Nitric Oxide Production in the Macrophage Cell Line RAW264.7 and Their Structure-Activity Relationships. Biochemical Pharmacology 1999; 58: 759-765.

[15] Theoharides T C. Method of treating mast cell activation-induced diseases with a proteoglycan. U.S. Pat. No. 6, 689,748 B1 2004.

[16] Akiyama T, Ishida J, Nakagawa S, Ogawara H, Watanabe S, Itoh N, Shibuya M, Fukami Y. Genistein a specific inhibitor of tyrosine-specificprotein kinase. Journal of Biological Chemistry 1987; 262: 5592-5595

[17] Nichols M R, Morimoto B H. Tyrosine kinase-independent inhibition of cyclic-AMP phosphodiesterase by Genistein and Tyrphostin 51. Archives of Biochemistry and Biophysics 1999; 366: 224-230

[18] Nichols M R, Morimoto B H. Differential inhibition of multiple cAMP phosphodiesterase isozymes by isoflavones and tyrphostins. Molecular Pharmacology 2000; 57: 738-745

[19] Underwood D C, Osborn R R, Novak L B, Matthews J K, Newsholme S J, Undem B J, Hand J M, Torphy T J. Inhibition of antigen-induced bronchoconstriction and eosinophil infiltration in the guinea pig by the cyclic AMP-specific phosphodiesterase inhibitor, rolipram. Journal of Pharmacology and Experimental Therapeutics 1993; 266: 306-313

[20] Underwood D C, Kotzer C J, Bochnowicz S, Osborn R R, Luttmann M A, Hay D W, Torphy T J. Comparison of phosphodiesterase III, IV and dual III/IV inhibitors on bronchospasm and pulmonary eosinophil influx in guinea pigs. Journal of Pharmacology and Experimental Therapeutics 1994; 270: 250-259

[21] Compton C H, Gubb J, Nieman R, Edelson J, Amit O, Bakst A, Ayres J G, Creemers J P, Schultze-Werninghaus G, Brambilla C, and Barnes N C. Cilomilast, a selective phosphodiesterase-4 inhibitor for treatment of patients with chronic obstructive pulmonary disease: a randomised, dose-ranging study. Lancet 2001; 358: 265-270.

[22] Barnette M S, Bartus J O, Burman M, Christensen S B, Cieslinski L B, Esser K M, Prabhakar U S, Rush J A, and Torphy T J. Association of the anti-inflammatory activity of phosphodiesterase 4 (PDE4) inhibitors with either inhibition of PDE4 catalytic activity or competition for [³H]rolipram binding. Biochem. Pharmacol. 51: 949-956, 1996.

[23] Schneider H H, Schmiechen R, Brezinski M, and Seidler J. Stereospecific binding of the antidepressant rolipram to brain protein structures. European Journal of Pharmacology 1986; 127: 105-115.

[24] Hatzelmann A and Schudt C. Anti-inflammatory and immunomodulatory potential of the novel PDE4 inhibitor roflumilast in vitro. Journal of Pharmacology and Experimental Therapeutics 2001; 297: 267-279.

[25] Zhao Y, Zhang H T, and O'Donnell J M. Inhibitor binding to type 4 phosphodiesterase (PDE4) assessed using [3H]piclamilast and [3H]rolipram. Journal of Pharmacology and Experimental Therapeutics 2003; 305: 565-572.

[26] Draheim R, Egerland U, and Rundfeldt C. Anti-inflammatory potential of the selective phosphodiesterase 4 inhibitor N-(3,5-dichloro-pyrid-4-yl)-[1-(4-fluorobenzyl)-5-hydroxy-indole-3-yl]-gly oxylic acid amide (AWD 12-281), in human cell preparations. Journal of Pharmacology and Experimental Therapeutics 2004; 308: 555-563.

[27] Ko W C, Shih C M, Lai Y H, Chen J H, and Huang H L. Inhibitory effects of flavonoids on phosphodiesterase isozymes from guinea pig and their structure-activity relationships. Biochemical Pharmacology 2004; 68: 2087-2094.

[28] Ko W C, Chen M C, Wang S H, Lai Y H, Chen J H, Lin C N. 3-O-methylquercetin more selectively inhibits phosphodiesterase subtype 3. Planta medica 2003; 69: 310-315

[29] Thompson W J, Appleman M M. Multiple cyclic nucleotide phosphodiesterase activities from rat brain. Biochemistry 1971; 10: 311-316

[30] Ahn H S, Crim W, Romano M, Sybertz E, Pitts B. Effects of selective inhibitors on cyclic nucleotide phosphodiesterases of rabbit aorta. Biochemical Pharmacology 1989; 38: 3331-3339

[31] Podzuweit T, Nennstiel P, and Muller A, Isozyme selective inhibition of cGMP-stimulated cyclic nucleotide phosphodiesterases by erythro-9-(2-hydroxy-3-nonyl)adenine. Cellular Signalling 7: 733-738, 1995.

[32] Harrison S A, Reifsnyder D H, Gallis B, Cadd G G, Beavo J A. Isolation and characterization of bovine cardiac muscle cGMP-inhibited phosphodiesterase: a receptor for new cardiotonic drugs. Molecular Pharmacology 1986; 29: 506-514

[33] Reeves M L, Leigh B K, and England P J, The identification of a new cyclic nucleotide phosphodiesterase activity in human and guinea-pig cardiac ventricle. Implications for the mechanism of action of selective phosphodiesterase inhibitors. Biochemical Journal 241: 535-541, 1987.

[34] Gillespie P G, Beavo J A. Inhibition and stimulation of photoreceptor phosphodiesterases by dipyridamole and M&B 22,948. Molecular Pharmacology 1989; 36: 773-781

[35] Bradford M M. A rapid and sensitive method for quantitation of microgram quantities of protein utilizing the principle of protein-dye binding. Analytical Biochemistry 1976; 72: 248-254

[36] Kanehiro A, Ikemura T, Mäkelä M J, Lahn M, Joetham A, Dakhama A, Gelfand E W. Inhibition of phosphodiesterase 4 attenuates airway hyperresponsiveness and airway inflammation in a model of secondary allergen challenge. American Journal of Respiratory and Critical Care Medicine 2001; 163: 173-184

[37] Hamelmann E, Schwarze J, Takeda K, Oshiba A, Larsen G L, Irvin C G, Gelfand E W. Noninvasive measurement of airway responsiveness in allergic mice using barometric plethysmography. American Journal of Respiratory and Critical Care Medicine 1997; 156: 766-775

What is claimed is:

1. A medical composition comprising:
a flavonoid as an active constituent selectively inhibiting at least one of a phosphodiesterase 4 and a phosphodiesterase 3/4, and represented by the formula (I): 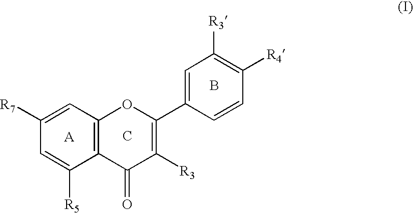
wherein,
R₃, R₅ and R₇ are ones selected from the group consisting of hydrogen, hydroxyl, benzyl, phenyl, phenyl having a substituent being one selected form the group consisting of hydroxyl, O-acyl-R₈, ethoxyl, n-propoxyl, i-propoxyl, n-butoxyl and i-butoxyl, and benzyl having a substituent being one selected form the group consisting of hydroxyl, O-acyl-R₈, ethoxyl, n-propoxyl, i-propoxyl, n-butoxyl and i-butoxyl,
respective R_{3′} and R_{4′} are ones selected from the group consisting of hydrogen, hydroxyl, O-acyl-R₈, ethoxyl, n-propoxyl, i-propoxyl, n-butoxyl, i-butoxyl, and
R₈ is one selected from the group consisting of methyl, ethyl, propyl, n-butyl, and i-butyl.

2. The medical composition according to claim 1, wherein the flavonoid is one selected from the group consisting of luteolin, luteolin-7-glucoside, diosmetin, apigenin, chrysin, quercetin, and myricetin.

3. The medical composition according to claim 1 having an effect of one of treating a chronic inflammation and being as a bronchodilator.

4. The medical composition according to claim 1 further comprising one of a medical excipient or thinner.

5. A medical composition comprising:
a flavonoid as an active constituent selectively inhibiting at least one of a phosphodiesterase 4 and a phosphodiesterase 3/4 and represented by the formula (II): 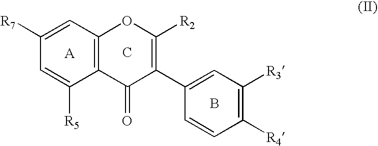
wherein,
R₂, R₅ and R₇ are ones selected from the group consisting of hydrogen, hydroxyl, benzyl, phenyl, phenyl having a substituent being one selected form the group consisting of hydroxyl, O-acyl-R₈, ethoxyl, n-propoxyl, i-propoxyl, n-butoxyl and i-butoxyl, and benzyl having a substituent being one selected form the group consisting of hydroxyl, O-acyl-R₈, ethoxyl, n-propoxyl, i-propoxyl, n-butoxyl and i-butoxyl,
respective R_{3′} and R_{4′} are ones selected from the group consisting of hydrogen, hydroxyl, O-acyl-R₈, ethoxyl, n-propoxyl, i-propoxyl, n-butoxyl, i-butoxyl, and
R₈ is one selected from a group consisting of methyl, ethyl, propyl, n-butyl, and i-butyl.

6. The medical composition according to claim 5, wherein the flavonoid is one selected from the group consisting of genistein, daidzein, biochanin A, and prunetin.

7. The medical composition according to claim 5 having an effect of one of treating a chronic inflammation and being as a bronchodilator.

8. The medical composition according to claim 5 further comprising one of a medical excipient or thinner.

9. A medical composition comprising:
a flavonoid as an active constituent selectively inhibiting at least one of a phosphodiesterase 4 and a phosphodiesterase 3/4, and represented by the formula (III): 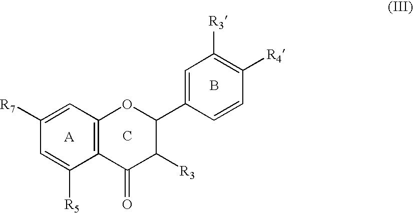
wherein,
the respective R₃, R₅ and R₇ are ones selected from the group consisting of hydrogen, hydroxyl, benzyl, phenyl, phenyl having a substituent being one selected form the group consisting of hydroxyl, O-acyl-R₈, ethoxyl, n-propoxyl, i-propoxyl, n-butoxyl and i-butoxyl, and benzyl having a substituent being one selected form the group consisting of hydroxyl, O-acyl-R₈, ethoxyl, n-propoxyl, i-propoxyl, n-butoxyl and i-butoxyl,
respective R_{3′} and R_{4′} are ones selected from the group consisting of hydrogen, hydroxyl, O-acyl-R₈, ethoxyl, n-propoxyl, i-propoxyl, n-butoxyl, i-butoxyl, and
R₈ is one selected from a group consisting of methyl, ethyl, propyl, n-butyl, and i-butyl.

10. The medical composition according to claim 9, wherein the flavonoid is one of eriodictyol or hesperetin.

11. The medical composition according to claim 9 having an effect of one of treating a chronic inflammation and being as a bronchodilator.

12. The medical composition according to claim 9 further comprising one of a medical excipient or thinner.
